(19) United States
(12) Patent Application Publication
(10) Pub. No.: US 2026/0179290 A1
(43) Pub. Date: Jun. 25, 2026

(54) IMAGE RECONSTRUCTION METHOD AND RADIONUCLIDE DEVICE IMAGING METHOD

(71) Applicant: Shanghai United Imaging Healthcare Co., Ltd., Shanghai (CN)

(72) Inventors: Yihuan Lu, Shanghai (CN); Duo Zhang, Shanghai (CN)

(21) Appl. No.: 19/433,953

(22) Filed: Dec. 28, 2025

(30) Foreign Application Priority Data
Dec. 25, 2024 (CN) 202411932785.6

(51) Int. Cl. G06T 12/10; A61B 6/03; G01T 1/29; G06T 7/11; G06T 7/215; G06T 12/20

(52) U.S. Cl. CPC G06T 12/10; A61B 6/037; G01T 1/2985; G06T 7/11; G06T 7/215; G06T 12/20; G06T 2207/10104; G06T 2207/20021; G06T 2207/20084; G06T 2211/441

(57) ABSTRACT

An image reconstruction method includes: obtaining scanning data of a scanned object; obtaining motion information corresponding to one or more local regions of the scanned object respectively, based on the scanning data and movement information of a scan bed; and performing reconstruction with correction on the scanning data based on the motion information to obtain a reconstructed image of the scanned object.

### CROSS-REFERENCE TO RELATED APPLICATIONS

The present application claims priority to Chinese patent application No. 2024119327856, filed on Dec. 25, 2024, and titled “Image Reconstruction Method and Radionuclide Device Imaging Method”, the entire content of which is incorporated herein by reference.

### TECHNICAL FIELD

The present disclosure relates to the field of medical technologies, and in particular, to an image reconstruction method and a radionuclide device imaging method.

### BACKGROUND

Since the axial field-of-view (FOV) length of most Positron Emission Computed Tomography (PET) systems cannot fully cover the whole-body area of a patient, a “multi-bed scanning” mode is usually adopted during PET scanning. That is, PET data are acquired multiple times when the scan bed is at different positions, so that the acquired PET data can sufficiently cover the patient's whole-body area, and the PET data from multi-bed scanning are stitched into a complete PET image during reconstruction. However, this method greatly increases the overall scanning time, and the “scan-table shift-scan” mode will increase the patient's discomfort caused by intermittent movement and stopping.

To address the problems of long scanning time and patient discomfort caused by “multi-bed scanning”, existing solutions mainly adopt the “continuous table-moving scanning” mode, but in this mode, it is difficult to achieve high-quality image reconstruction for key regions.

### SUMMARY

An image reconstruction method and a radionuclide device imaging method are provided in the present disclosure.

In a first aspect, an image reconstruction method is provided in the present disclosure, including:
obtaining scanning data of a scanned object, where the scanning data include first scanning data and second scanning data, the first scanning data are acquired by a scanning device when a scan bed carrying the scanned object is controlled to be stationary, and the second scanning data are acquired by the scanning device when the scan bed is moving;
obtaining motion information corresponding to one or more local regions of the scanned object respectively, based on the scanning data and movement information of the scan bed; and
performing reconstruction with correction on the scanning data based on the motion information to obtain a reconstructed image of the scanned object.

In some embodiments, the method further includes:
obtaining a real-time position of the scan bed acquired by a sensor; and
determining the movement information based on a difference between the detection position of the scanning device and the real-time position of the scan bed.

In some embodiments, obtaining the motion information corresponding to the one or more local regions of the scanned object based on the scanning data and the movement information of the scan bed includes:
performing segmentation processing on a pre-obtained to-be-segmented image of the scanned object to obtain a plurality of local segmentation masks of the scanned object;
determining the one or more local regions based on the movement information and the plurality of local segmentation masks, where the one or more local regions are within a field of view of the scanning device; and
obtaining the motion information corresponding to the one or more local regions based on the one or more local regions and the scanning data corresponding to the one or more local regions, respectively.

In some embodiments, the plurality of local segmentation masks includes segmentation labels for the one or more local regions of the scanned object.

In some embodiments, performing segmentation processing on the pre-obtained to-be-segmented image of the scanned object to obtain the plurality of local segmentation masks of the scanned object includes: adopting a segmentation network or an image segmentation algorithm to perform segmentation processing on the to-be-segmented image to obtain the plurality of local segmentation masks of the scanned object.

In some embodiments, obtaining the motion information corresponding to the one or more local regions based on the one or more local regions and the scanning data corresponding to the one or more local regions, respectively, includes:
for each of the one or more local regions, performing analysis on the scanning data corresponding to the one or more local regions based on a centroid-based motion analysis method to obtain preliminary motion information corresponding to the one or more local regions; and
converting the preliminary motion information corresponding to the one or more local regions to a same standard to obtain the motion information corresponding to the one or more local regions.

In some embodiments, the first scanning data correspond to a target region of the scanned object, the second scanning data correspond to a region of interest of the scanned object, and performing reconstruction with correction on the scanning data based on the motion information to obtain a reconstructed image of the scanned object includes:
processing the scanning data to obtain sub-scanning data; and
performing, based on the motion information, reconstruction with correction on at least one set of the sub-scanning data, which correspond to the motion information, to obtain the reconstructed image, the at least one set of the sub-scanning data including the scanning data corresponding to the target region.

In some embodiments, processing the scanning data to obtain the sub-scanning data includes: dividing the scanning data based on at least one of the motion information or timestamp information of the scanning data to obtain the sub-scanning data. Each set of the sub-scanning data correspond to different local regions or time periods.

In some embodiments, obtaining the scanning data of the scanned object includes:
obtaining the first scanning data acquired by the scanning device for the target region when the scan bed is controlled to be stationary; and
obtaining the second scanning data acquired by the scanning device for the region of interest when the scan bed is moving.

In a second aspect, radionuclide device imaging method is further provided in the present disclosure, which is applied to a scanning system. The scanning system includes a radionuclide device and a scan bed. The method includes:
controlling the scan bed to carry the scanned object to move from one end of the radionuclide device to a center of a field of view of the radionuclide device at a predetermined moving speed, and acquiring first scanning data;
pausing the movement of the scan bed and acquiring second scanning data, in response to a center of a region of interest of the scanned object carried by the scan bed moving to the center of the field of view; and
performing reconstruction based on the first scanning data and the second scanning data to obtain a reconstructed image of the scanned object, a resolution of the region of interest in the reconstructed image being higher than a resolution of other regions.

In some embodiments, performing reconstruction based on the first scanning data and the second scanning data to obtain a reconstructed image of the scanned object includes:
determining sub-scanning data corresponding to one or more local regions of the scanned object based on the movement information of the scan bed, the first scanning data, and the second scanning data;
obtaining motion information corresponding to the one or more local regions based on the sub-scanning data corresponding to one or more local regions; and
performing reconstruction with correction on each set of the sub-scanning data based on the motion information to obtain the reconstructed image of the scanned object.

In some embodiments, the method further includes:
obtaining a real-time position of the scan bed acquired by a sensor; and
determining the movement information based on a difference between a detection position of a scanning device and the real-time position of the scan bed.

In some embodiments, determining the sub-scanning data corresponding to the one or more local regions of the scanned object based on the movement information of the scan bed, the first scanning data, and the second scanning data includes:
performing segmentation processing on a pre-obtained to-be-segmented image of the scanned object to obtain a plurality of local segmentation masks of the scanned object;
determining the one or more local regions based on the movement information and the plurality of local segmentation masks, where the one or more local regions are within a field of view of the scanning device; and
determining the sub-scanning data corresponding to the one or more local regions of the scanned object based on each of the one or more local regions, the first scanning data, and the second scanning data.

In some embodiments, obtaining the motion information corresponding to the one or more local regions based on each set of the sub-scanning data corresponding to each of the one or more local regions includes:
for each of the one or more local regions, performing analysis on the sub-scanning data corresponding to the one or more local regions based on a centroid-based motion analysis method to obtain preliminary motion information corresponding to the one or more local regions; and
converting the preliminary motion information corresponding to the one or more local regions to a same standard to obtain the motion information corresponding to the one or more local regions.

In a third aspect, a radionuclide device imaging method is provided in the present disclosure, which is applied to a scanning system. The scanning system includes a radionuclide device and a scan bed, and the method includes:
controlling the scan bed to carry a scanned object to move intermittently, and controlling the radionuclide device to continuously obtain scanning data of the scanned object during intermittent movement of the scan bed;
obtaining movement information of the scan bed during acquisition of the scanning data;
obtaining motion information corresponding to one or more local regions of the scanned object respectively, based on the scanning data and the movement information of the scan bed; and
performing reconstruction with correction on the scanning data based on the motion information to obtain a reconstructed image of the scanned object.

In some embodiments, scanning of a target region of the scanned object is performed during pause intervals of the intermittent movement, and scanning is performed on at least regions of the scanned object excluding the target region during a movement phase of the intermittent movement. The reconstructed image is a whole-body image of the scanned object.

In some embodiments, obtaining the movement information of the scan bed includes:
obtaining a real-time position of the scan bed acquired by a sensor; and
determining the movement information based on a difference between a detection position of a scanning device and the real-time position of the scan bed.

In some embodiments, obtaining the motion information corresponding to the one or more local regions of the scanned object based on the scanning data and the movement information of the scan bed includes:
performing segmentation processing on a pre-obtained to-be-segmented image of the scanned object to obtain a plurality of local segmentation masks of the scanned object;
determining the one or more local regions based on the movement information and the plurality of local segmentation masks, where the one or more local regions are within a field of view of the scanning device; and
obtaining the motion information corresponding to the one or more local regions based on the one or more local regions and the scanning data corresponding to the one or more local regions, respectively.

In some embodiments, obtaining the motion information corresponding to the one or more local regions based on the one or more local regions and the scanning data corresponding to the one or more local regions, respectively, includes:
for each of the one or more local regions, performing analysis on the scanning data corresponding to the one or more local regions based on a centroid-based motion analysis method to obtain preliminary motion information corresponding to the one or more local regions; and
converting the preliminary motion information corresponding to the one or more local regions to a same standard to obtain the motion information corresponding to the one or more local regions.

In a fourth aspect, an image reconstruction apparatus is further provided in the present disclosure, including:
a first acquisition module configured to obtain scanning data of a scanned object, where the scanning data include first scanning data and second scanning data, the first scanning data are acquired by a scanning device when a scan bed carrying the scanned object is controlled to be stationary, and the second scanning data are acquired by the scanning device when the scan bed is moving;
a second acquisition module configured to obtain motion information corresponding to one or more local regions of the scanned object respectively, based on the scanning data and movement information of the scan bed; and
a first reconstruction module configured to perform reconstruction with correction on the scanning data based on the motion information to obtain a reconstructed image of the scanned object.

In a fifth aspect, a computer device is further provided in the present disclosure, including a memory and a processor, the memory storing a computer program. The processor, when executing the computer program, implements steps of the method in the first aspect, the second aspect, and the third aspect.

In a sixth aspect, a computer-readable storage medium having a computer program stored thereon is further provided in the present disclosure. The computer program, when executed by a processor, causes the processor to implement the steps of the method of the first aspect, the second aspect, and the third aspect.

In a seventh aspect, a computer program product is further provided in the present disclosure, including a computer program. The computer program, when executed by a processor, causes the processor to implement steps of the method in the first aspect, the second aspect, and the third aspect.

### BRIEF DESCRIPTION OF THE DRAWINGS

To more clearly illustrate the technical solutions in the embodiments of the present disclosure or related technologies, accompanying drawings required for describing the embodiments of the present disclosure or related technologies will be briefly introduced below. Apparently, the accompanying drawings described below are only some embodiments of the present disclosure, and those of ordinary skill in the art can obtain other related drawings based on these drawings without creative effort.

FIG. 1 is a schematic diagram of an application environment of an image reconstruction method in some embodiments of the present disclosure.

FIG. 2A is a schematic flowchart of an image reconstruction method in some embodiments of the present disclosure.

FIG. 2B is a schematic flowchart of an image reconstruction method in other embodiments of the present disclosure.

FIG. 3 is a schematic flowchart of a method for determining movement information in some embodiments of the present disclosure.

FIG. 4A is a schematic flowchart of a method for determining motion information in some embodiments of the present disclosure.

FIG. 4B is a schematic flowchart of a method for determining motion information in other embodiments of the present disclosure.

FIG. 5A is a schematic flowchart of a method for determining motion information in other embodiments of the present disclosure.

FIG. 5B is a schematic flowchart of a method for determining motion information in other embodiments of the present disclosure.

FIG. 6 is a schematic flowchart of an image reconstruction method in other embodiments of the present disclosure.

FIG. 7 is a block diagram of a configuration of an image reconstruction apparatus in some embodiments of the present disclosure.

### DETAILED DESCRIPTION OF THE EMBODIMENTS

The technical solutions in the embodiments of the present disclosure will be clearly and completely described below with reference to the accompanying drawings in the embodiments of the present disclosure. Apparently, the described embodiments are only a part of the embodiments of the present disclosure, rather than all the embodiments. Based on the embodiments in the present disclosure, all other embodiments obtained by those of ordinary skill in the art without creative effort shall fall within the protection scope of the present disclosure.

In the related art, when performing continuous table-moving scanning, the patient will move with a scan bed to pass through a PET detector. During this process, the scan bed will move continuously without pausing, and a movement distance of the scan bed exceeds the axial FOV (axial field-of-view) length of the detector to cover an entire area of the patient. During the continuous movement of the patient with the scan bed, the PET detector continuously acquires PET coincidence event data, records each line of response (LOR), and stores data as list-mode data for subsequent image reconstruction. However, in clinical PET scanning, regions of interest vary across different departments, different doctors, and different patients. A continuous table-moving technology cannot well focus on key regions of interest. Although different scan bed moving speeds can be used to achieve scanning of different regions, low speed cannot break through physical limits of scan bed movement to achieve near-static slow table-moving. Therefore, the present disclosure proposes an image reconstruction method and a radionuclide device imaging method that can solve the above technical problems.

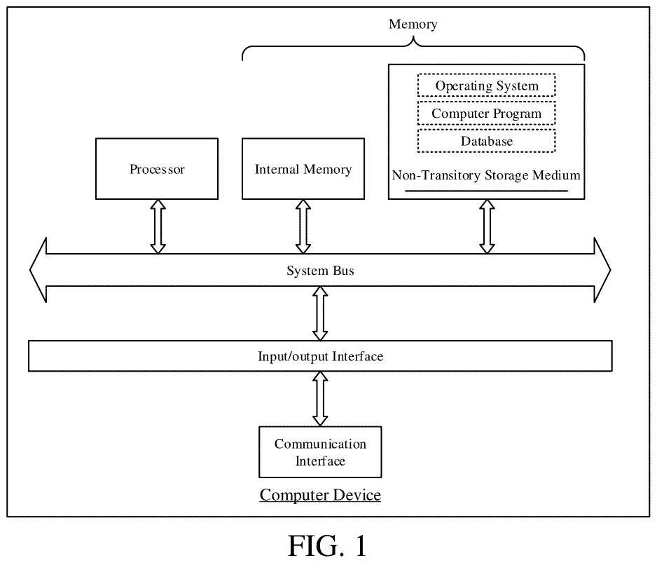

The image reconstruction method provided in the embodiment of the present disclosure can be applied to an application environment shown in FIG. 1. The application environment includes a computer device, which may be a server, and its internal configuration can be as shown in FIG. 1. The computer device includes a processor, a memory, an Input/Output (I/O) interface, and a communication interface. The processor, memory, and I/O interface are connected through a system bus, and the communication interface is connected to the system bus through the I/O interface. The processor of the computer device is configured to provide computing and control capabilities. The memory of the computer device includes a non-transitory storage medium and an internal memory. The non-transitory storage medium stores an operating system, a computer program, and a database. The internal memory provides an environment for the operation of the operating system and the computer program in the non-transitory storage medium. The database of the computer device is configured to store data related to image reconstruction. The I/O interface of the computer device is configured to exchange information between the processor and external devices. The communication interface of the computer device is configured to communicate with an external terminal through a network connection. The computer program is executed by the processor to implement an image reconstruction method. The server may be an independent physical server, a server cluster, or a distributed system composed of multiple physical servers, or a cloud server providing cloud computing services.

Those skilled in the art can understand that the configuration shown in FIG. 1 is only a block diagram of a part of the configuration related to the solution of the present disclosure, and does not constitute a limitation on the computer device to which the solution of the present disclosure is applied. A specific computer device may include more or fewer components than those shown in FIG. 1, or combine some components, or have different component arrangements.

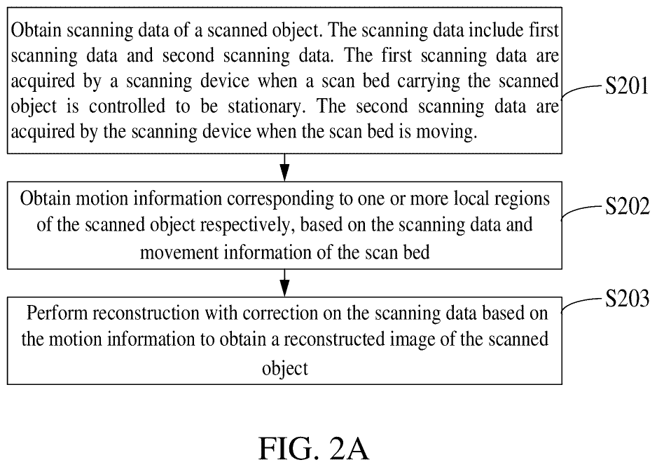

In an exemplary embodiment, as shown in FIG. 2A, an image reconstruction method is provided, which is described by taking the method applied to the computer device in FIG. 1 as an example, including the following steps S201 to S203.

In step S201, scanning data of a scanned object are obtained. The scanning data include first scanning data and second scanning data. The first scanning data are acquired by a scanning device when a scan bed carrying the scanned object is controlled to be stationary. The second scanning data are acquired by the scanning device when the scan bed is moving.

Optionally, the first scanning data may be scanning data corresponding to a target region of the scanned object, for example, a key region of the scanned object concerned by a doctor. The second scanning data may be scanning data corresponding to a region of interest of the scanned object. The scan bed may be controlled to be stationary for a predetermined duration, and the scanning device is controlled to scan a target region of the scanned object to obtain the first scanning data. The scan bed may be controlled to move at a predetermined moving speed, and the scanning device is controlled to scan the region of interest of the scanned object to obtain the second scanning data. The predetermined moving speed may be fixed or variable. The first scanning data enables static high-resolution scanning for the target region, while the second scanning data enables continuous mobile coverage scanning for non-target regions. All scanning data are stored in association with the movement information of the scan bed, facilitating subsequent correction during reconstruction.

Optionally, the first scanning data may be scanning data corresponding to a target region of the scanned object acquired when the scan bed is stationary, and the second scanning data may be scanning data acquired when the scan bed is moving (which may cover the region of the scanned object excluding the target region or include a partial region of the target region). The scanning data can be whole-body data of the scanned object. When the scan bed performs reverse movement (e.g., being pulled out of the detector aperture) to return to an initial position, even if the target region is within the field of view during the reverse movement, the scanning data acquired in this phase still belongs to the second scanning data. This is because the second scanning data is defined by the moving state of the scan bed, regardless of the movement direction or whether the target region is covered.

In this embodiment, taking the target region and the region of interest as examples, the scan bed is controlled to be stationary at the target region, and the scanning device performs data acquisition on the target region to obtain the first scanning data. In the region of interest excluding the target region, the scan bed is controlled to move, and data acquisition is performed on the region of interest to obtain the second scanning data.

Optionally, the scanning device may be a detector in PET imaging, and the scanning data may be all PET photon coincidence event data detected during this period, including detector matrix array information corresponding to the events, detected photon energy information, timestamp information of the coincidence events, etc.

Optionally, the scanning device may scan the target region and the region of interest only when the scan bed enters the FOV. It may also scan the target region and the region of interest when the scan bed enters and exits the FOV to obtain the first scanning data and the second scanning data, i.e., the scan bed performs a reciprocating motion. The scan bed may also repeat the reciprocating motion for multiple times, and during this repetition, the scanning device continuously scans the target region and the region of interest to obtain the first scanning data and the second scanning data. For example, when the detector starts scanning, the scan bed carries the scanned object to be pushed into an aperture of the detector at a predetermined speed, the detector continuously acquires PET photon coincidence event data and transmits it to the computer device, and the computer device stores the received photon coincidence event data. When the center of the target region is at the center position of the detector, the scan bed stops moving for a predetermined duration, the detector continuously acquires photon coincidence event data, and the computer device stores the scanning data. After the scanning in the stationary state of the scan bed is completed, the scan bed continues to be pushed in at a predetermined speed, the PET detector continuously acquires PET photon coincidence event data and transmits it to the computer device, and the computer device stores the received photon coincidence event data in the storage medium of the computer device until an center of the region of interest reaches the center of the PET detector. The scan bed stops moving briefly and is pushed out in a reverse direction at a predetermined speed, the PET detector continuously acquires PET photon coincidence event data and transmits it to the computer device, and the computer device stores the received photon coincidence event data in the storage medium of the computer device until the scan bed returns to the starting position.

Optionally, the scan bed may stop being pushed in when the center of the region of interest reaches the center or any other point of the PET detector, including but not limited to its edges or any position along its interior. The starting position may be a position where the center of the region of interest leaves the edge of the detector, or a position where the scanned object is completely outside the FOV of the detector.

Optionally, the predetermined moving speed may be constant, or different predetermined speeds may be set according to clinical requirements of each sub-region in the region of interest. For example, the target region is a heart, the scanning duration of the heart is set to 5 seconds, and the region of interest may be other body regions excluding the heart, or other body regions of the upper body excluding the heart, or limbs, etc. A speed of the region of interest close to the heart is less than that of the region of interest far from the heart.

Optionally, a maximum moving distance of the scan bed refers to a distance that the scan bed needs to move to enable the scanning device to cover the region of interest and the target region. For a same device, the maximum moving distance of the scan bed may differ depending on different scanning protocols. The maximum moving distance may exceed an axial FOV length of the scanning device or be shorter than the axial FOV length of the scanning device.

In this embodiment, after the scanning device acquires the scanning data of the scanned object, the scanning data may be transmitted to the computer device in real time, or the scanning data transmitted by the scanning device may be stored in the computer device. The computer device obtains the scanning data from the storage medium of the computer when receiving an instruction, or periodically receives the scanning data transmitted by the scanning device.

FIG. 2B is a schematic diagram of a scanning device performing scanning on a scanned object in some embodiments of the present disclosure. The axial field of view (FOV) formed by the PET detectors of the scanning device arranged side by side along an axial direction has a length of 120 cm. The scanned object is in a supine position with feet first, and the target region is an upper abdomen of the patient. During a period from when lower limbs of the scanned object carried by the scan bed start to enter a FOV range to when the target region is moved to the center of the FOV, the PET detector of the scanning device is controlled to continuously acquire the second scanning data. When the center of the target region is at the center position of the detector, the scan bed pauses intermittently, the detector continuously acquires photon coincidence event data, and the computer device stores the scanning data to obtain the first scanning data. At this time, the first scanning data may include large parts such as the head, upper abdomen, lower abdomen to knee joint. After the scanning and imaging of the target region is completed, the scan bed carries the scanned object to exit from the FOV (head first). The PET detector of the scanning device is controlled to continuously acquire the second scanning data again.

Optionally, when the scope of the key region exceeds the axial field of view (FOV) length of the detector, the detector continuously acquires second scanning data for non-key regions during the movement of the scan bed carrying the scanned object. When the center of any sub-region of the key region moves to the center of the field of view, the scan bed pauses to acquire high-resolution first scanning data of the sub-region. The movement acquisition and stationary acquisition can be repeated. After covering all sub-regions of the key region, all data are integrated for reconstruction and correction by combining the movement information of the scan bed and the motion information extracted by the centroid method, so as to achieve complete high-resolution imaging of the key region and whole-body coverage.

Optionally, for two or more discontinuous key regions, after predetermining the scanning sequence and parameters, the scan bed drives the scanned object to move towards a first key region, and the second scanning data for non-key regions are synchronously acquired. When the center of the key region reaches the center of the field of view, the scan bed pauses and the first scanning data are acquired. Subsequently, the scan bed moves to the next key region and repeats the above process until all key regions are scanned. The data of each region are associated through the movement information of the scan bed, and corrected in combination with the motion information, so as to ensure the imaging quality of each discontinuous key region and realize whole-body scanning coverage.

In step S202, motion information corresponding to one or more local regions of the scanned object are obtained respectively, based on the scanning data and movement information of the scan bed.

In this embodiment, a real-time position of the scan bed during the acquisition of the scanning data acquired by a sensor may be obtained, and the movement information of the scan bed is determined based on the difference between the detection position of the scanning device and the real-time position of the scan bed. The detection position of the scanning device refers to a spatial position of a core detection component (such as the center of the annular aperture of the PET detector) in the scanning device for acquiring scanning data, which can be used to establish an origin reference of a scanning coordinate system. The movement information may include the moving direction, speed, stationary state, etc. of the scan bed. A reference position used to determine the movement information include, but are not limited to, the detection position of the scanning device; for example, it may also be an edge of the detector, a calibrated position of a specific detection unit, etc.

Further, the motion information corresponding to the one or more local regions during the movement of the scan bed may be obtained based on the movement information of the scan bed and the scanning data. During the acquisition of scanning data, a corresponding relationship between each set of scanning data and a scan bed code is obtained. Each PET detector ring is assigned an index number, the position of the PET detector is fixed, and the scan bed code is a known parameter. During the movement of the scan bed, the corresponding scan bed code is acquired simultaneously with each set of scanning data. Based on aforementioned corresponding relationships, a plurality of sets of scanning data can be reclassified into a plurality of scanning data sets. Each scanning data set includes a plurality of sets of scanning data corresponding to a same scanning region at different time points. Specifically, the scanning region of the scanned object can be accurately positioned based on the scan bed code. Combined with the corresponding relationship between the scanning data and the bed code, the scanning region to which each set of scanning data belongs can be clarified, and then the scanning data belonging to the same scanning region are grouped into one scanning data set. At least one scanning data set is a collection of coincidence events acquired by detector rings within different intermittent time periods.

The motion information may include local body motion signals, such as respiratory signals, heartbeat signals, head motion signals, body motion signals, etc. Since the scanning data are acquired during the scanning process of the scanned object carried by the scan bed, the movement information of the scan bed can refer to the motion information corresponding to one or more local regions during the movement of the scan bed. When the scanned object moves with the scan bed, the positions of its local regions change in real time with the scan bed. By obtaining the movement information of the scan bed, the change can be dynamically tracked to obtain the motion information corresponding to the one or more local regions, so as to ensure the accurate correspondence between the one or more local regions and the scanning data.

In step S203, reconstruction with correction is performed on the scanning data based on the motion information to obtain a reconstructed image of the scanned object.

In this embodiment, based on the motion information corresponding to one or more local regions of the scanned object, the scanning data are processed, and reconstruction with correction may be performed on each of the one or more local regions of the scanned object respectively. For example, the scanning data may be divided to obtain sub-scanning data corresponding to local regions, the position information in the sub-scanning data are corrected based on the motion information corresponding to the local region, and the corrected sub-scanning data are reconstructed to obtain a reconstructed image. The reconstructed image includes multiple anatomical structures, and the resolution of at least one anatomical structure in the reconstructed image is higher than the resolution of other regions.

In a possible implementation, any one piece of motion information may be used as the target motion information, and the target motion information is used to perform reconstruction with correction on the scanning data to obtain a reconstructed image of the scanned object.

In the above image reconstruction method, scanning data of a scanned object are obtained. Motion information corresponding to one or more local regions of the scanned object during the movement of the scanned object along with the scan bed is obtained, based on the scanning data and the movement information of the scan bed. Reconstruction with correction is performed on the scanning data based on the motion information to obtain a reconstructed image of the scanned object. The scanning data include first scanning data and second scanning data. The first scanning data are acquired by a scanning device when a scan bed carrying the scanned object is controlled to be stationary. The second scanning data are acquired by the scanning device when the scan bed is moving. The motion information is determined based on the movement information of the scan bed. In the embodiment of the present disclosure, scanning is performed in a stop-movement combined scanning mode by controlling the scan bed to be stationary and move, which can effectively improve the scanning efficiency on the basis of full coverage of the scanning region of the scanned object. In addition, by controlling the scan bed to be in a stationary state during the scanning process, more sufficient scanning data can be acquired and the imaging quality of the reconstructed image of the corresponding region can be improved.

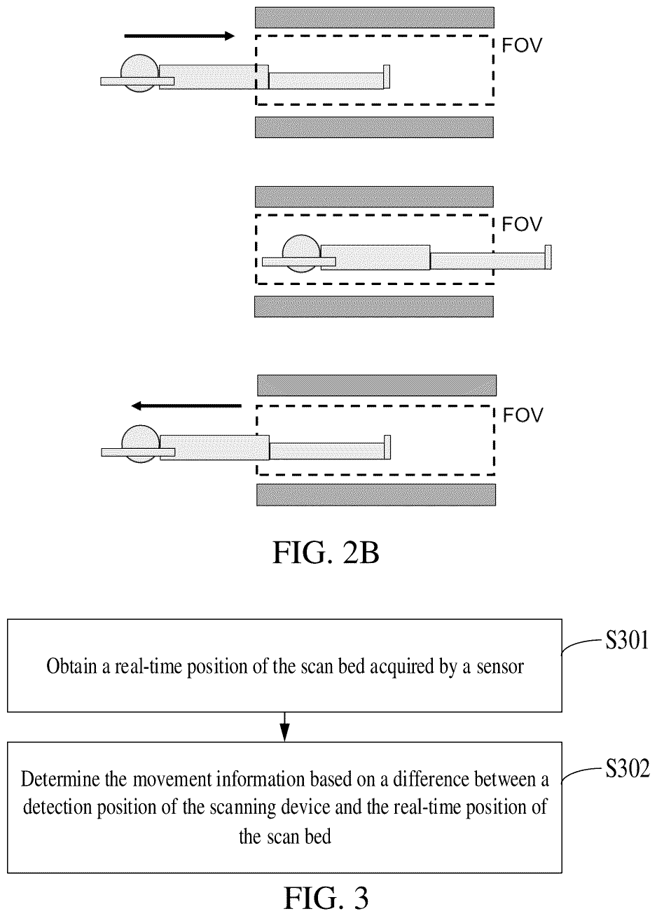

FIG. 3 is a schematic flowchart of a method for determining movement information in some embodiments. As shown in FIG. 3, the method includes the following steps S301 to S302.

In step S301, a real-time position of the scan bed acquired by a sensor is obtained.

In this embodiment, a position sensor may be installed on the scan bed. When the scanning device acquires scanning data, the position sensor is configured to acquire the real-time position of the scan bed in real time, and the real-time position of the scan bed is transmitted to the computer device for simultaneous recording and tracking, and stored together with the scanning data.

In step S302, the movement information is determined based on a difference between a detection position of the scanning device and the real-time position of the scan bed.

The movement information refers to the motion information corresponding to the scanned object and the scan bed as a whole.

In this embodiment, for the scanning device, the scan bed drives the scanned object to move all the time, and due to the continuous movement of the scan bed, the motion information corresponding to the scanned object is increased. A three-dimensional Cartesian coordinate system is established with the detection position of the scanning device as an origin of the coordinate system. The detection position of the scanning device may be a center of the effective FOV of the PET detector. Initial position coordinates of the position sensor on the scan bed in the coordinate system may be determined based on the detection position of the scanning device. When the scan bed moves, the coordinates of the position sensor will change accordingly, i.e., the coordinates of the position sensor can reflect the difference between the detection position of the scanning device and the real-time position of the scan bed, and the movement information of the scan bed is obtained based on the change of the coordinates of the position sensor.

In the embodiment of the present disclosure, the real-time position of the scan bed acquired by the sensor is obtained, and the movement information is determined based on the difference between the detection position of the scanning device and the real-time position of the scan bed. In conventional technologies, continuous table-moving scanning increases the difficulty of obtaining motion information by a data-driven method during the scanning process. In the present disclosure, the target region is scanned in a single-bed scanning mode, and scanning is continuously performed during the movement of the scan bed to cover the region of interest. The method can effectively extract motion information of each local region, which is then used for motion correction during image reconstruction, thereby improving the image quality of the reconstructed image.

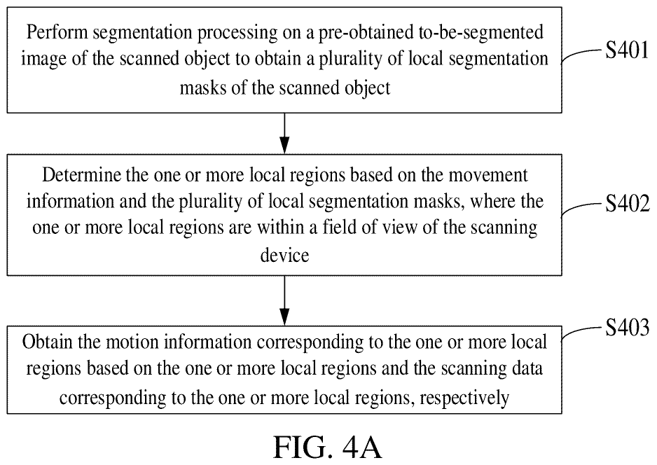

FIG. 4A is a schematic flowchart of a method for determining motion information in some embodiments. As shown in FIG. 4A, obtaining the motion information corresponding to the one or more local regions of the scanned object based on the scanning data and the movement information of the scan bed includes the following steps S401 to S403.

In step S401, segmentation processing is performed on a pre-obtained to-be-segmented image of the scanned object to obtain a plurality of local segmentation masks of the scanned object.

Optionally, the to-be-segmented image of the scanned object may be a CT image acquired by a PET/Computed Tomography (CT) system, an MRI image of a PET/Magnetic Resonance Imaging (MRI) system, a PET reconstructed image, or a PET direct back-projection image.

In some embodiments, the segmentation processing may adopt various segmentation forms, and the local segmentation masks may be segmentation labels of multiple organs of the scanned object, segmentation labels of any part of the body, or segmentation labels of simple segmentation based on axial regions.

In this embodiment, a segmentation network may be adopted to perform segmentation processing on the to-be-segmented image to obtain a plurality of local segmentation masks of the scanned object, or an image segmentation algorithm may be adopted to perform segmentation processing on the to-be-segmented image to obtain a plurality of local segmentation masks of the scanned object. For example, a binarization algorithm may be adopted.

In step S402, the one or more local regions are determined based on the movement information and the plurality of local segmentation masks, where the one or more local regions are within a field of view of the scanning device.

In this embodiment, since the FOV of the scanning device cannot fully cover the scanned object, different body regions are within the FOV of the scanning device at different times during the movement of the scan bed. Assuming that the position sensor is installed at the front end of the scan bed, a positional relationship between each of the one or more local regions can be obtained based on each of the local segmentation masks, and the relationship between the movement information and the FOV of the scanning device (the edge position close to the scan bed side) is obtained by comparison to determine at least one of local regions within the FOV of the scanning device.

In step S403, the motion information corresponding to the one or more local regions are obtained based on the one or more local regions and the scanning data corresponding to the one or more local regions, respectively.

The motion information can be local body motion signals, such as respiratory signals, heartbeat signals, head motion signals, body motion signals, etc.

In this embodiment, for each of the one or more local regions, the scanning data corresponding to the one or more local regions may be analyzed based on a centroid-based motion analysis method to obtain preliminary motion information corresponding to the one or more local regions, and the preliminary motion information is used as the motion information. For example, for each of the one or more local regions, centroid coordinates are obtained from the corresponding scanning data based on the centroid-based motion analysis method. The centroid coordinates corresponding to each moment are calculated, and the respiratory signals corresponding to the one or more local regions are obtained with time as the horizontal axis and the centroid coordinates as the vertical axis. The respiratory signals are used as the motion information corresponding to the one or more local regions.

In some embodiments, a plurality of PET images corresponding to the scanning region may be pre-generated for a plurality of data frames, and one PET image is generated based on one data frame. Subsequently, a motion curve of the scanning region is obtained based on the plurality of PET images, and the motion curve may include a centroid motion curve of a specific organ (e.g., the heart) of the scanned object. The horizontal coordinates represent a time period (for example, the time period is 1.5 minutes, and a time unit is a nth 100 microseconds, where n is a positive integer), and the vertical coordinates represent relative positions of centroids of the PET images corresponding to the scanning region.

Optionally, the motion information may be overall direction signals obtained in the x/y/z three directions, or signals in any one of the x/y/z directions.

FIG. 4B is a schematic diagram of a method for determining motion information in some embodiments. Three organ markers or segmentation masks of kidney, liver and heart are set for the scanned object, and motion information extraction is performed only when the corresponding organ markers or segmentation masks are detected to be within the FOV. During a period from when the lower limbs of the scanned object carried by the scan bed start to enter the FOV range to when the target region is moved to the center of the FOV, the kidney and liver are first detected to enter the FOV range, and then the motion information corresponding to the kidney and liver can be extracted. While the heart is outside the FOV, the motion information corresponding to the heart is not extracted at this time. When the center of the target region is at the center position of the detector, the scan bed pauses intermittently, and at this time, the three organs of kidney, liver and heart are within the FOV, and the motion information corresponding to the three organs can be acquired simultaneously.

In the embodiment of the present disclosure, segmentation processing is performed on a pre-obtained to-be-segmented image to obtain a plurality of local segmentation masks of the scanned object. At least one of local regions within the FOV of the scanning device is determined based on the movement information and the plurality of local segmentation masks. For each of the one or more local regions, the scanning data corresponding to the one or more local regions are analyzed based on a centroid-based motion analysis method to obtain the motion information corresponding to the one or more local regions. Since there are also involuntary movements of the scanned object during the movement of the scan bed, such as breathing, heartbeat, head movement, body movement, etc., in the embodiment of the present disclosure, a plurality of local segmentation masks can be identified by a data-driven method, the one or more local regions within the FOV can be determined based on the plurality of local segmentation masks and movement information, and the motion information corresponding to the one or more local regions can be extracted combined with the centroid analysis method, laying a foundation for subsequent reconstruction with correction of the scanning data based on the motion information.

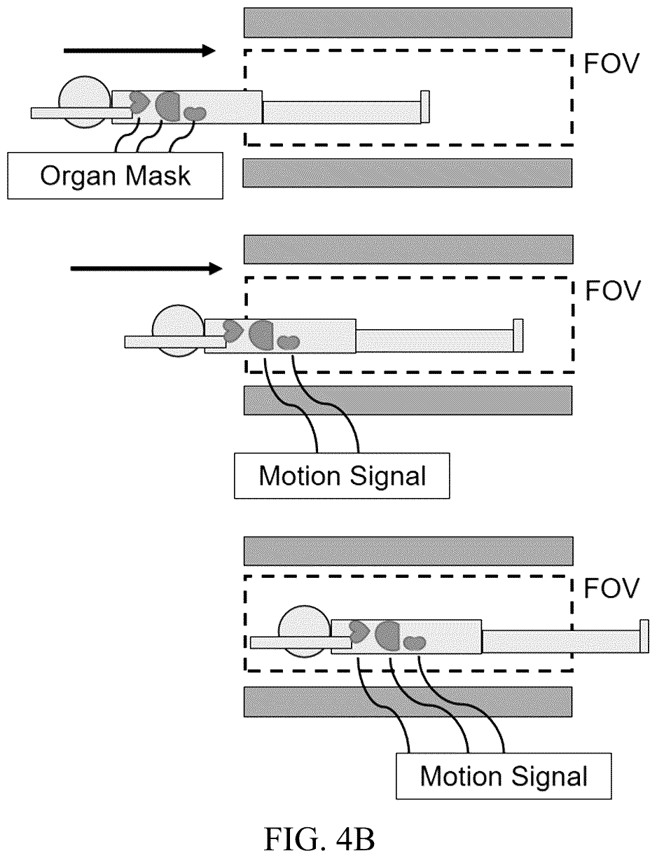

FIG. 5A is a schematic flowchart of a method for determining motion information in other embodiments. As shown in FIG. 5A, in the embodiment of the present disclosure, obtaining the motion information corresponding to the one or more local regions based on the one or more local regions and the scanning data corresponding to the one or more local regions, respectively, includes the following steps S501 to S502.

In step S501, for each of the one or more local regions, analysis is performed on the scanning data corresponding to the one or more local regions based on a centroid-based motion analysis method to obtain preliminary motion information corresponding to the one or more local regions.

In step S502, the preliminary motion information corresponding to the one or more local regions are converted to a same standard to obtain the motion information corresponding to the one or more local regions.

In this embodiment, a convolution method or a signal correlation analysis method may be adopted to convert the preliminary motion information to the same standard to obtain converted body motion information. Alternatively, normalization is used to convert the preliminary motion information to the same standard to obtain converted motion information. For example, the preliminary motion information includes heartbeat signals of a heart and respiratory signals of a lung, a variation range of the heartbeat signals of the heart is 0 to 10, and a variation range of the respiratory signals of the lung is 0 to 5. The heartbeat signals of the heart may be divided by 2, and the converted signals and the respiratory signals of the lung have the same variation range. Alternatively, the heartbeat signals of the heart may be divided by 10, the respiratory signals of the lung may be divided by 5, and both the heartbeat signals of the heart and the respiratory signals of the lung are converted to the range of 0 to 1.

FIG. 5B is a schematic diagram of motion information in another embodiment. Referring to the schematic diagram shown in FIG. 4B, the kidney enters the FOV first, so longest motion information can be obtained. Then, the kidney and liver enter the FOV, and the motion information corresponding to the kidney and liver can be extracted at this time. Finally, three organs of kidney, liver and heart are within the FOV, and the motion information corresponding to the three organs can be acquired simultaneously. The three organs have motion information acquired at the same time (as shown by the dashed box in FIG. 5B). Correlation analysis can be performed on the motion information acquired at the same time, and consistency processing can be performed on the motion information corresponding to the three organs based on the correlation analysis results.

In the embodiment of the present disclosure, for each of the one or more local regions, the scanning data corresponding to the one or more local regions are analyzed based on a centroid-based motion analysis method to obtain preliminary motion information corresponding to the one or more local regions. The preliminary motion information is converted to the same standard to obtain the motion information. In the embodiment of the present disclosure, the preliminary motion information is converted to the same standard to determine the motion information, which can prevent the transition of the preliminary motion information.

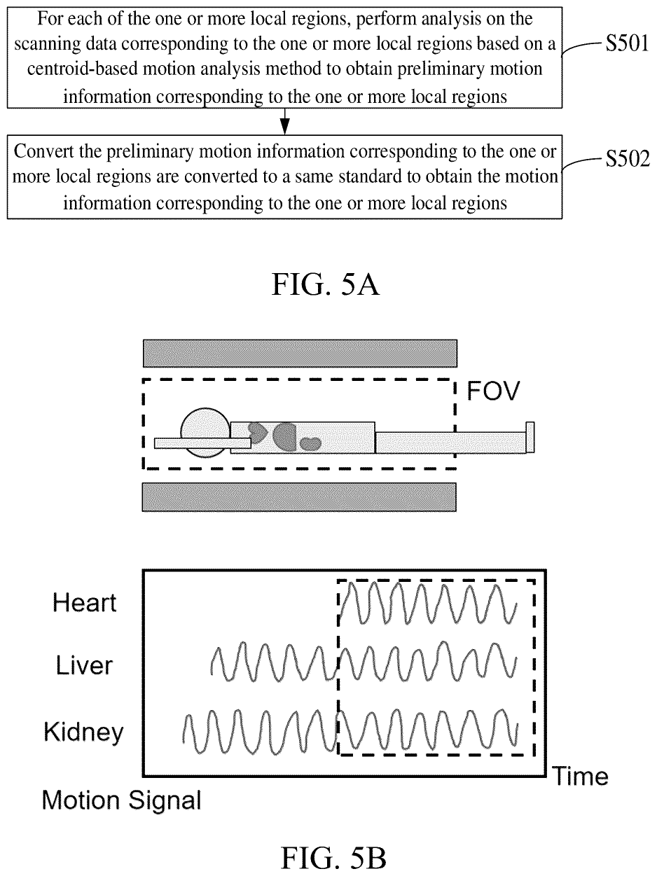

FIG. 6 is a schematic flowchart of an image reconstruction method in another embodiment, as shown in FIG. 6. The embodiment of the present disclosure relates to a possible implementation of how to perform reconstruction with correction on the scanning data based on the motion information to obtain a reconstructed image of the scanned object, including the following steps S601 to S602.

In step S601, the scanning data are divided to obtain the sub-scanning data.

The first scanning data correspond to a target region of the scanned object, and the second scanning data correspond to a region of interest of the scanned object.

In this embodiment, since the scanning device can also obtain time information corresponding to the scanning data while acquiring the scanning data, such as a timestamp, as a timing identifier recorded simultaneously when the scanning data are acquired. The scanning data can be divided into sub-scanning data corresponding to a plurality of time periods based on timestamps. The motion information corresponding to one or more local regions of the scanned object during the movement of the scanned object along with the scan bed may also be obtained, and the scanning data are divided into sub-scanning data corresponding to a plurality of local regions based on the motion information. In some examples, the motion information may also reflect a time period corresponding to the motion signal (such as breathing, heartbeat) of the local region. For example, a time period when the lung is within the FOV, a diastole or systole phase of the heartbeat signal, and these region-related motion information can be used as a basis for segmenting the scanning data based on local regions or physiological phases.

In a possible implementation, the corrected scanning data may also be arbitrarily divided to obtain sub-scanning data.

In step S602, reconstruction with correction is performed on at least one set of the sub-scanning data, which correspond to the motion information, based on the motion information to obtain the reconstructed image. The at least one set of the sub-scanning data includes the scanning data corresponding to the target region.

In this embodiment, an image reconstruction algorithm is used to reconstruct at least one set of the sub-scanning data, and at the same time, positions corresponding to the sub-scanning data are corrected in combination with the motion information corresponding to the sub-scanning data to obtain a reconstructed image of the scanned object.

In a possible implementation, the target motion information may be determined based on the motion information, an image reconstruction algorithm is used to reconstruct at least one set of the sub-scanning data, and at the same time, the positions corresponding to the sub-scanning data are corrected in combination with the target motion information to obtain a reconstructed image of the scanned object.

In the embodiment of the present disclosure, the scanning data are divided to obtain sub-scanning data, and at least one set of the sub-scanning data are reconstructed in combination based on the motion information to obtain a reconstructed image. In the embodiment of the present disclosure, the scanning data are divided based on the motion information or timestamp information, and data corresponding to different time periods are reconstructed in segments, or any axial region of the patient can be selected for selective reconstruction, which improves the flexibility in image processing and analysis.

In some embodiments, a radionuclide device imaging method is provided, which is applied to a scanning system; the scanning system includes a radionuclide device and a scan bed. The scan bed can be configured to perform reciprocating motion between two axial ends of the radionuclide device. The method includes: controlling the scan bed to carry the scanned object to move from one end of the radionuclide device to a center of a field of view of the radionuclide device at a predetermined moving speed, and acquiring first scanning data, pausing the movement of the scan bed and acquiring second scanning data, in response to a center of a region of interest of the scanned object carried by the scan bed moving to the center of the field of view, and performing reconstruction based on the first scanning data and the second scanning data to obtain a reconstructed image of the scanned object, a resolution of the region of interest in the reconstructed image being higher than a resolution of other regions.

Specifically, when the scan bed drives the scanned object to move in a continuous movement manner, the radionuclide device can simultaneously and continuously acquire scanning data. When the scan bed drives the scanned object to move in an intermittent movement method, the radionuclide device can perform intermittent acquisition to obtain scanning data correspondingly, i.e., during the movement phase of the scan bed, the radionuclide device simultaneously acquires scanning data corresponding to non-target regions. During pause intervals of the scan bed, the radionuclide device acquires scanning data corresponding to the target region to ensure the imaging quality of key region and realize effective coverage and image reconstruction of the whole-body region of the scanned object.

The scan bed is configured to carry the scanned object to move continuously or intermittently.

In some embodiments, performing reconstruction based on the first scanning data and the second scanning data to obtain a reconstructed image of the scanned object includes: determining sub-scanning data corresponding to one or more local regions of the scanned object based on the movement information of the scan bed, the first scanning data, and the second scanning data, obtaining motion information corresponding to the one or more local regions based on the sub-scanning data corresponding to one or more local regions, and performing reconstruction with correction on each set of the sub-scanning data based on the motion information to obtain the reconstructed image of the scanned object.

In this embodiment, the scan bed may carry the scanned object to move from one end of the radionuclide device to the other end, or may move back and forth multiple times between the two ends of the radionuclide device. During the movement, the scanning data of the scanned object are obtained, and the sub-scanning data corresponding to the one or more local regions of the scanned object are determined based on the movement information of the scan bed and the scanning data. For each of the one or more local regions, the corresponding motion information is obtained based on the sub-scanning data corresponding to the one or more local regions, the position information in the corresponding sub-scanning data is corrected based on the motion information, and the corrected scanning data are reconstructed to obtain a reconstructed image. Continuous movement may mean that the scan bed carries the scanned object to move continuously at a predetermined speed (for example, a fixed speed or a variable speed) without active pause during the movement, and the radionuclide device can simultaneously and continuously acquire scanning data. Intermittent movement may mean that the scan bed carries the scanned object to move in a cycle mode of movement-pause-movement. For example, during the pause intervals, the scan bed is controlled to be stationary, and the radionuclide device can acquire scanning data corresponding to the target region of the scanned object. During the movement phase of the scan bed, the radionuclide device can cover other regions excluding the target region and simultaneously acquire scanning data. In the embodiment of the present disclosure, scanning is performed in a single-bed stop-movement combined manner, which improves the reconstruction quality of the reconstructed image while improving the scanning efficiency.

In some embodiments, a radionuclide device imaging method is provided, which is applied to a scanning system. The scanning system includes a radionuclide device and a scan bed. The method includes: controlling the scan bed to carry a scanned object to move intermittently, and controlling the radionuclide device to continuously obtain scanning data of the scanned object during intermittent movement of the scan bed, obtaining movement information of the scan bed during acquisition of the scanning data, obtaining motion information corresponding to one or more local regions of the scanned object respectively, based on the scanning data and the movement information of the scan bed, and performing reconstruction with correction on the scanning data based on the motion information to obtain a reconstructed image of the scanned object.

Scanning of a target region of the scanned object is performed during pause intervals of the intermittent movement, and scanning is performed on at least regions of the scanned object excluding the target region is performed during the movement phase of the intermittent movement. The reconstructed image is a whole-body image of the scanned object.

In this embodiment, the target region of the scanned object is scanned during the pause intervals, and the regions excluding the target region are scanned during the movement phase of the scan bed to obtain the whole-body scanning data of the scanned object. Meanwhile, during the acquisition of the scanning data, the movement information of the scan bed is obtained, and the motion information corresponding to the one or more local regions are obtained based on the scanning data and the movement information. The scanning data are corrected by using the motion information, and reconstruction is performed based on corrected scanning data to obtain a reconstructed image. In the embodiment of the present disclosure, the motion information corresponding to one or more local regions are obtained by a data-driven method to provide motion correction information during image reconstruction, which improves the image quality of the reconstructed image.

It should be understood that although the various steps in the flowcharts involved in the above embodiments are displayed in sequence according to the arrows, these steps are not necessarily executed in sequence according to the order indicated by the arrows. Unless explicitly stated herein, there is no strict order restriction on the execution of these steps, and these steps can be executed in other orders. Moreover, at least some of the steps in the flowcharts involved in the above embodiments may include multiple steps or multiple stages, which are not necessarily executed at the same time, but can be executed at different times, and the execution order of these steps or stages is not necessarily sequential, but can be performed alternately or alternately with at least part of the steps or stages in other steps or other steps.

Based on the same inventive concept, the embodiment of the present disclosure further provides an image reconstruction apparatus for implementing the above-mentioned image reconstruction method. The solution provided by the apparatus for solving the problem is similar to the implementation solution recorded in the above method, so the specific limitations in one or more embodiments of the image reconstruction apparatus provided below can be referred to the limitations on the image reconstruction method above, and details are not repeated here.

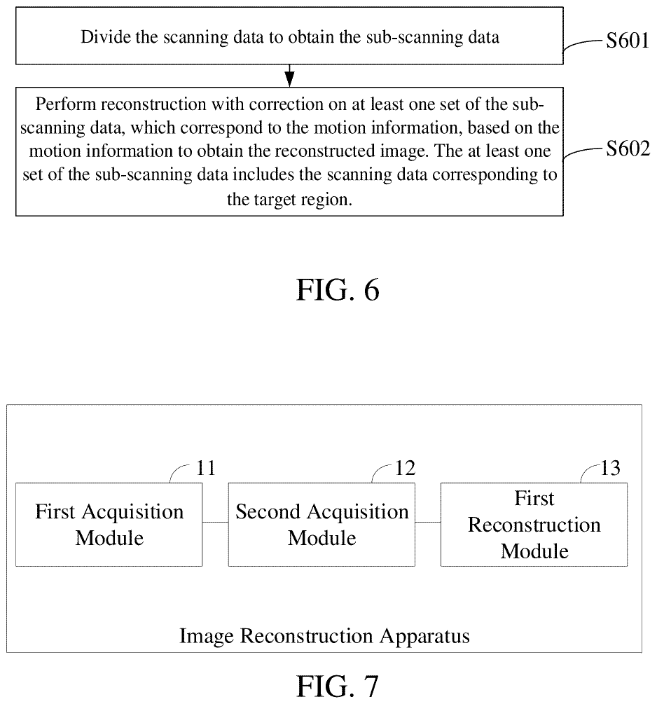

In an exemplary embodiment, as shown in FIG. 7, an image reconstruction apparatus is provided, including a first acquisition module 11, a second acquisition module 12, and a first reconstruction module 13.

The first acquisition module 11 is configured to obtain scanning data of a scanned object. The scanning data include first scanning data and second scanning data. The first scanning data are acquired by a scanning device when a scan bed carrying the scanned object is controlled to be stationary. The second scanning data are acquired by the scanning device when the scan bed is moving.

The second acquisition module 12 is configured to obtain motion information corresponding to one or more local regions of the scanned object respectively, based on the scanning data and movement information of the scan bed.

The first reconstruction module 13 is configured to perform reconstruction with correction on the scanning data based on the motion information to obtain a reconstructed image of the scanned object.

In some embodiments, the apparatus further includes a third acquisition module and a first determination module.

The third acquisition module is configured to obtain the real-time position of the scan bed acquired by a sensor.

The first determination module is configured to determine the movement information based on a difference between a detection position of the scanning device and the real-time position of the scan bed.

In some embodiments, the second acquisition module 12 is configured to perform segmentation processing on a pre-obtained to-be-segmented image of the scanned object to obtain a plurality of local segmentation masks of the scanned object, determine the one or more local regions based on the movement information and the plurality of local segmentation masks, where the one or more local regions are within a field of view of the scanning device, and obtain the motion information corresponding to the one or more local regions based on the one or more local regions and the scanning data corresponding to the one or more local regions, respectively.

In some embodiments, the second acquisition module 12 is further configured to adopt a segmentation network or an image segmentation algorithm to perform segmentation processing on the to-be-segmented image to obtain the plurality of local segmentation masks of the scanned object.

In some embodiments, the second acquisition module 12 is configured to, for each of the one or more local regions, perform analysis on the scanning data corresponding to the one or more local regions based on a centroid-based motion analysis method to obtain preliminary motion information corresponding to the one or more local regions, and convert the preliminary motion information corresponding to the one or more local regions to a same standard to obtain the motion information corresponding to the one or more local regions.

In some embodiments, the first reconstruction module 13 is configured to process the scanning data to obtain sub-scanning data, and perform reconstruction with correction on at least one of the plurality of sets of sub-scanning data, which correspond to the motion information, based on the motion information, to obtain the reconstructed image. The at least one set of the sub-scanning data includes the scanning data corresponding to the target region.

In some embodiments, the first reconstruction module 13 is further configured to divide the scanning data based on at least one of the motion information or timestamp information of the scanning data to obtain the sub-scanning data. Each set of the sub-scanning data correspond to different local regions or time periods.

In some embodiments, the first reconstruction module 13 is further configured to obtain the first scanning data acquired by the scanning device for the target region when the scan bed is controlled to be stationary, and obtain the second scanning data acquired by the scanning device for the region of interest when the scan bed is moving.

In some embodiments, a radionuclide device imaging apparatus is further provided, which is applied to a scanning system. The scanning system includes a radionuclide device and a scan bed, and the scan bed is configured to perform reciprocating motion between two axial ends of the radionuclide device. The apparatus includes a first control module, a second determination module, a fifth acquisition module, and a second reconstruction module.

The first control module is configured to obtain scanning data of a scanned object and movement information of the scan bed. The scanning data of the scanned object are continuously or intermittently acquired during movement of the scan bed.

The second determination module is configured to determine sub-scanning data corresponding to one or more local regions of the scanned object based on the movement information of the scan bed and the scanning data.

The fifth acquisition module is configured to obtain motion information corresponding to the one or more local regions based on each set of the sub-scanning data corresponding to each of the one or more local regions.

The second reconstruction module is configured to perform reconstruction with correction on each set of the sub-scanning data based on the motion information to obtain a reconstructed image of the scanned object.

In some embodiments, the scan bed is configured to carry the scanned object to move continuously or intermittently.

In some embodiments, the apparatus further includes a third determination module. The third determination module is configured to obtain a real-time position of the scan bed acquired by a sensor, and determine the movement information based on a difference between a detection position of a scanning device and the real-time position of the scan bed.

In some embodiments, the second determination module is further configured to perform segmentation processing on a pre-obtained to-be-segmented image of the scanned object to obtain a plurality of local segmentation masks of the scanned object, determine the one or more local regions based on the movement information and the plurality of local segmentation masks, where the one or more local regions are within a field of view of the scanning device, and determine the sub-scanning data corresponding to the one or more local regions of the scanned object based on each of the one or more local regions and the scanning data.

In some embodiments, the fifth acquisition module is further configured to, for each of the one or more local regions, perform analysis on the sub-scanning data corresponding to the one or more local regions based on a centroid-based motion analysis method to obtain preliminary motion information corresponding to the one or more local regions, and convert the preliminary motion information corresponding to the one or more local regions to a same standard to obtain the motion information corresponding to the one or more local regions.

In some embodiments, a radionuclide device imaging apparatus is further provided, which is applied to a scanning system. The scanning system includes a radionuclide device and a scan bed. The apparatus includes a second control module, a sixth acquisition module, a seventh acquisition module, and a third reconstruction module.

The second control module is configured to control the scan bed to carry a scanned object to move intermittently, and control the radionuclide device to continuously obtain scanning data of the scanned object during intermittent movement of the scan bed.

The sixth acquisition module is configured to obtain movement information of the scan bed during acquisition of the scanning data.

The seventh acquisition module is configured to obtain motion information corresponding to one or more local regions of the scanned object respectively, based on the scanning data and the movement information of the scan bed.

The third reconstruction module is configured to perform reconstruction with correction on the scanning data based on the motion information to obtain a reconstructed image of the scanned object.

In some embodiments, the sixth acquisition module is further configured to obtain a real-time position of the scan bed acquired by a sensor, and determine the movement information based on a difference between a detection position of a scanning device and the real-time position of the scan bed.

In some embodiments, the seventh acquisition module is further configured to perform segmentation processing on a pre-obtained to-be-segmented image of the scanned object to obtain a plurality of local segmentation masks of the scanned object, determine the one or more local regions based on the movement information and the plurality of local segmentation masks, where the one or more local regions are within a field of view of the scanning device, and obtain the motion information corresponding to the one or more local regions based on the one or more local regions and the scanning data corresponding to the one or more local regions, respectively.

In some embodiments, the seventh acquisition module is further configured to, for each of the one or more local regions, perform analysis on the scanning data corresponding to the one or more local regions based on a centroid-based motion analysis method to obtain preliminary motion information corresponding to the one or more local regions, and convert the preliminary motion information corresponding to the one or more local regions to a same standard to obtain the motion information corresponding to the one or more local regions.

Each module in the above image reconstruction apparatus can be implemented in whole or in part by software, hardware, or a combination thereof. The above modules can be embedded in or independent of the processor in the computer device in the form of hardware, or stored in the memory in the computer device in the form of software, so that the processor can call and execute the operations corresponding to the above modules.

In an exemplary embodiment, a computer device is provided, including a memory and a processor. The memory stores a computer program, and the processor implements the steps of any of the above method embodiments when executing the computer program.

In some embodiments, a computer-readable storage medium is provided, on which a computer program is stored. The computer program, when executed by a processor, causes the processor to implement the steps of any of the above method embodiments.

In some embodiments, a computer program product is provided, including a computer program, when executed by a processor, causes the processor to implement the steps of any of the above method embodiments.

It should be noted that the user information (including but not limited to user device information, user personal information, etc.) and data (including but not limited to data for analysis, stored data, displayed data, etc.) involved in present disclosure are all information and data authorized by the user or fully authorized by all parties, and the acquisition, use and processing of relevant data need to comply with relevant regulations.

Those of ordinary skill in the art can understand that all or part of the processes in the above embodiment methods can be completed by instructing relevant hardware through a computer program, and the computer program can be stored in a non-transitory computer-readable storage medium. When the computer program is executed, it may include the processes of the above method embodiments. Any reference to memory, database or other media in the various embodiments provided in present disclosure may include at least one of non-transitory memory and volatile memory. Non-transitory memory may include read-only memory (ROM), magnetic tape, floppy disk, flash memory, optical memory, high-density embedded non-transitory memory, resistive random access memory (ReRAM), magnetoresistive random access memory (MRAM), ferroelectric random access memory (FRAM), phase change memory (PCM), graphene memory, etc. Volatile memory may include random access memory (RAM) or external cache memory, etc. As an illustration but not a limitation, RAM can be in various forms, such as static random access memory (SRAM) or dynamic random access memory (DRAM). The databases involved in the various embodiments provided in present disclosure may include at least one of relational databases and non-relational databases. Non-relational databases may include blockchain-based distributed databases, etc., without limitation. The processors involved in the various embodiments provided in present disclosure may be general-purpose processors, central processing units, graphics processors, digital signal processors, programmable logic devices, quantum computing-based data processing logic devices, artificial intelligence (AI) processors, etc., without limitation.

The technical features of the above embodiments can be combined arbitrarily. To make the description concise, all possible combinations of the technical features in the above embodiments are not described. However, as long as there is no contradiction in the combination of these technical features, all of them should be considered as the scope of the description in present disclosure.

The above embodiments only represent several implementation modes of the present disclosure, and their descriptions are specific and detailed, but they should not be construed as limiting the scope of the patent of the present disclosure. It should be pointed out that for those of ordinary skill in the art, without departing from the concept of the present disclosure, several modifications and improvements can be made, which all belong to the protection scope of the present disclosure. Therefore, the protection scope of the present disclosure should be subject to the appended claims.

What is claimed is:

1. An image reconstruction method, comprising:
obtaining scanning data of a scanned object, wherein the scanning data comprise first scanning data and second scanning data, the first scanning data are acquired by a scanning device when a scan bed carrying the scanned object is controlled to be stationary, and the second scanning data are acquired by the scanning device when the scan bed is moving;
obtaining motion information corresponding to one or more local regions of the scanned object respectively, based on the scanning data and movement information of the scan bed; and
performing reconstruction with correction on the scanning data based on the motion information to obtain a reconstructed image of the scanned object.

2. The image reconstruction method according to claim 1, further comprising:
obtaining a real-time position of the scan bed acquired by a sensor; and
determining the movement information based on a difference between a detection position of the scanning device and the real-time position of the scan bed.

3. The image reconstruction method according to claim 1, wherein obtaining the motion information corresponding to the one or more local regions of the scanned object based on the scanning data and the movement information of the scan bed comprises:
performing segmentation processing on a pre-obtained to-be-segmented image of the scanned object to obtain a plurality of local segmentation masks of the scanned object;
determining the one or more local regions based on the movement information and the plurality of local segmentation masks, the one or more local regions being within a field of view of the scanning device; and
obtaining the motion information corresponding to the one or more local regions based on the one or more local regions and the scanning data corresponding to the one or more local regions, respectively.

4. The image reconstruction method according to claim 3, wherein the plurality of local segmentation masks comprises segmentation labels for the one or more local regions of the scanned object.

5. The image reconstruction method according to claim 3, wherein performing segmentation processing on the pre-obtained to-be-segmented image of the scanned object to obtain the plurality of local segmentation masks of the scanned object comprises:
adopting a segmentation network or an image segmentation algorithm to perform segmentation processing on the to-be-segmented image to obtain the plurality of local segmentation masks of the scanned object.

6. The image reconstruction method according to claim 3, wherein obtaining the motion information corresponding to the one or more local regions based on the one or more local regions and the scanning data corresponding to the one or more local regions, respectively, comprises:
for each of the one or more local regions, performing analysis on the scanning data corresponding to the one or more local regions based on a centroid-based motion analysis method to obtain preliminary motion information corresponding to the one or more local regions; and
converting the preliminary motion information corresponding to the one or more local regions to a same standard to obtain the motion information corresponding to the one or more local regions.

7. The image reconstruction method according to claim 1, wherein the first scanning data correspond to a target region of the scanned object, the second scanning data correspond to a region of interest of the scanned object, and performing reconstruction with correction on the scanning data based on the motion information to obtain the reconstructed image of the scanned object comprises:
processing the scanning data to obtain sub-scanning data; and
performing reconstruction with correction on at least one set of the sub-scanning data, which correspond to the motion information, based on the motion information, to obtain the reconstructed image, the at least one set of the sub-scanning data comprising the scanning data corresponding to the target region.

8. The image reconstruction method according to claim 7, wherein processing the scanning data to obtain the sub-scanning data comprises:
dividing the scanning data based on at least one of the motion information or timestamp information of the scanning data to obtain the sub-scanning data, each set of the sub-scanning data corresponding to different local regions or time periods.

9. The image reconstruction method according to claim 7, wherein obtaining the scanning data of the scanned object comprises:
obtaining the first scanning data acquired by the scanning device for the target region when the scan bed is controlled to be stationary; and
obtaining the second scanning data acquired by the scanning device for the region of interest when the scan bed is moving.

10. A radionuclide device imaging method, applied to a scanning system, the scanning system comprising a radionuclide device and a scan bed, wherein the method comprises:
controlling the scan bed to carry the scanned object to move from one end of the radionuclide device to a center of a field of view of the radionuclide device at a predetermined moving speed, and acquiring first scanning data;
pausing the movement of the scan bed and acquiring second scanning data, in response to a center of a region of interest of the scanned object carried by the scan bed moving to the center of the field of view; and
performing reconstruction based on the first scanning data and the second scanning data to obtain a reconstructed image of the scanned object, a resolution of the region of interest in the reconstructed image being higher than a resolution of other regions.

11. The radionuclide device imaging method according to claim 10, wherein performing reconstruction based on the first scanning data and the second scanning data to obtain a reconstructed image of the scanned object comprises:
determining sub-scanning data corresponding to one or more local regions of the scanned object based on the movement information of the scan bed, the first scanning data, and the second scanning data;
obtaining motion information corresponding to the one or more local regions based on the sub-scanning data corresponding to one or more local regions; and
performing reconstruction with correction on each set of the sub-scanning data based on the motion information to obtain the reconstructed image of the scanned object.

12. The radionuclide device imaging method according to claim 10, further comprising:
obtaining a real-time position of the scan bed acquired by a sensor; and
determining the movement information based on a difference between a detection position of a scanning device and the real-time position of the scan bed.

13. The radionuclide device imaging method according to claim 11, wherein determining the sub-scanning data corresponding to the one or more local regions of the scanned object based on the movement information of the scan bed, the first scanning data, and the second scanning data comprises:
performing segmentation processing on a pre-obtained to-be-segmented image of the scanned object to obtain a plurality of local segmentation masks of the scanned object;
determining the one or more local regions based on the movement information and the plurality of local segmentation masks, the one or more local regions being within a field of view of the scanning device; and
determining the sub-scanning data corresponding to the one or more local regions of the scanned object based on each of the one or more local regions, the first scanning data, and the second scanning data.

14. The radionuclide device imaging method according to claim 11, wherein obtaining the motion information corresponding to the one or more local regions based on each set of the sub-scanning data corresponding to each of the one or more local regions comprises:
for each of the one or more local regions, performing analysis on the sub-scanning data corresponding to the one or more local regions based on a centroid-based motion analysis method to obtain preliminary motion information corresponding to the one or more local regions; and
converting the preliminary motion information corresponding to the one or more local regions to a same standard to obtain the motion information corresponding to the one or more local regions.

15. A radionuclide device imaging method, applied to a scanning system, the scanning system comprising a radionuclide device and a scan bed, wherein the method comprises:
controlling the scan bed to carry a scanned object to move intermittently, and controlling the radionuclide device to continuously obtain scanning data of the scanned object during intermittent movement of the scan bed;
obtaining movement information of the scan bed during acquisition of the scanning data;
obtaining motion information corresponding to one or more local regions of the scanned object respectively, based on the scanning data and the movement information of the scan bed; and
performing reconstruction with correction on the scanning data based on the motion information to obtain a reconstructed image of the scanned object.

16. The radionuclide device imaging method according to claim 15, wherein scanning of a target region of the scanned object is performed during pause intervals of the intermittent movement, and scanning is performed on at least regions of the scanned object excluding the target region during a movement phase of the intermittent movement, and the reconstructed image is a whole-body image of the scanned object.

17. The radionuclide device imaging method according to claim 15, wherein obtaining the movement information of the scan bed comprises:
obtaining a real-time position of the scan bed acquired by a sensor; and
determining the movement information based on a difference between a detection position of a scanning device and the real-time position of the scan bed.

18. The radionuclide device imaging method according to claim 15, wherein obtaining the motion information corresponding to the one or more local regions of the scanned object based on the scanning data and the movement information of the scan bed comprises:
performing segmentation processing on a pre-obtained to-be-segmented image of the scanned object to obtain a plurality of local segmentation masks of the scanned object;
determining the one or more local regions based on the movement information and the plurality of local segmentation masks, the one or more local regions being within a field of view of the scanning device; and
obtaining the motion information corresponding to the one or more local regions based on the one or more local regions and the scanning data corresponding to the one or more local regions, respectively.

19. The radionuclide device imaging method according to claim 18, wherein obtaining the motion information corresponding to the one or more local regions based on the one or more local regions and the scanning data corresponding to the one or more local regions, respectively, comprises:
for each of the one or more local regions, performing analysis on the scanning data corresponding to the one or more local regions based on a centroid-based motion analysis method to obtain preliminary motion information corresponding to the one or more local regions; and
converting the preliminary motion information corresponding to the one or more local regions to a same standard to obtain the motion information corresponding to the one or more local regions.

20. A computer device, comprising a memory and a processor, the memory storing a computer program, wherein the processor, when executing the computer program, performs the image reconstruction method according to claim 1.
